(19) United States
(12) Patent Application Publication
(10) Pub. No.: US 2007/0073142 A1
(43) Pub. Date: Mar. 29, 2007

(54) Device and method for providing an angiographic image

(75) Inventors: Kai Eck, Aachen (DE); Jorg Bredno, Aachen (DE); Peter Rongen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V.

(21) Appl. No.: 10/575,572

(22) PCT Filed: Oct. 7, 2004
(86) PCT No.: PCT/IB04/52017

§ 371 Date: Apr. 11, 2006

(30) Foreign Application Priority Data
Oct. 16, 2003 (EP) 03103832.6

(51) Int. Cl. A61B 5/05

(52) U.S. Cl. 600/413

(57) ABSTRACT

The invention relates to a device and a method for providing an angiographic image (A) based on a database (2) with angiograms (3, 3a) from various heartbeat phases (H) and respiratory phases (R). From the available angiograms (3, 3a), a function (f) is first calculated, which describes the position (x) of the heart (1) dependent upon the respiratory phase (R). With the aid of this function (f) an angiographic image (A) may be generated which matches the heartbeat phase (H_{d}) and the respiratory phase (R_{d}) for given values of the heartbeat phase (H_{d}) and the respiratory phase (R_{d}), by transformation of an available angiogram (3a) which matches the heartbeat phase (H_{d}).

The invention relates to a device and a method for providing an angiographic image of a body structure matching a given heartbeat phase and respiratory phase.

For many medical operations on the vascular system of a patient, angiograms are needed. These are images of the vascular system on which the vessel courses are emphasized due, for instance, to the injection of a contrast medium. When, for instance, a catheter is pushed through the vascular system of a patient under fluoroscopic observation, angiograms may serve as static vessel maps in order to simplify navigation of the catheter and to minimize the loading of the patient with contrast medium.

Particularly when investigating the organs of the thoracic and abdominal cavities, the movement and deformation of the body structures due to the heartbeat and the breathing lead to the fact that current images of the vascular system only seldom match the stored static angiograms. For this reason, it is proposed, for instance in U.S. Pat. No. 6,473,635 B1, that angiograms stored in a database should be indexed according to their associated heartbeat phase and respiratory phase and that the respective angiogram should be selected for a representation together with a current fluoroscopic image whose parameters best match the current heartbeat phase/respiratory phase. A procedure of this type encounters problems, however, if no angiogram approximately matching the current phases is present in the database. The latter is relatively often the case, since the angiograms must cover a two-dimensional parameter region and, on the other hand, efforts are made to manage with the fewest possible angiograms to minimize the contrast medium loading.

Against this background, it is an object of the present invention to make available means for providing an angiographic image that matches a given heartbeat phase and respiratory phase.

This object is achieved by a device having the features of claim 1 and by a method having the features of claim 10. Advantageous embodiments are contained in the dependent claims.

The device according to the invention serves to provide an angiographic image of a body structure, such as the heart, whereby the angiographic image should in the best way possible match a given heartbeat phase and respiratory phase. The device includes a database (store) in which angiograms of the body structure in various heartbeat phases and respiratory phases are stored. The angiograms may be generated in conventional manner, for instance by X-ray projection imaging during a contrast-medium injection. The angiograms may also be two-dimensional or multi-dimensional. Typically, the database contains about 10 to 100, preferably approximately 30 to 50 angiograms. In what follows, the designation “angiogram” should preferably be used for images generated directly by an image-forming apparatus, while “angiographic image” may be either a directly generated or a calculated image.

The apparatus also contains a data processing apparatus linked to the database, arranged to carry out the following steps:

a) The calculation of a function which describes (at least) a change in the body structure occurring in the angiograms, dependent upon the respiratory phase, whereby said calculation takes place based on the angiograms in the database. The change in the body structure may in principle be any geometrical change, such as for instance, a displacement of the position of the body structure and/or a deformation of the body structure.

b) The generation of the angiographic image to be produced from such angiograms of the database, whose associated heartbeat phase matches the given heartbeat phase, whereby the generation takes place with the aid of the function calculated in step a).

With the described device, angiographic images may be provided which fit to a high degree of accuracy with a current heartbeat phase and respiratory phase. This achieves that (if necessary) the angiographic image is calculated from the existing angiograms, i.e. generated artificially. In this process, the fact is made use of that the change of the body structure due to the respiration takes place according to a functional pattern, which may be approximately determined from the data points present in the database.

The function calculated in step a) may optionally be limited to describing a pure change in the position of the body structure, i.e. a displacement and/or rotation. In many cases, the respiration has a negligible effect in the form of body structures, so that it substantially only brings about a positional change. In these cases, the generation of images in step b) is also correspondingly simplified, since for instance, it may be brought about by a corresponding positional change (displacement and/or rotation) of an angiogram which fits with the given heartbeat phase.

There are various possibilities for determining the change in position of a body structure. For instance, a prominent point on the body structure could be segmented in the angiograms and its positional change calculated. Preferably, however, for determining the positional change, a cross-correlation and/or a maximization of the mutual information is undertaken in relation to a reference angiogram.

According to a further embodiment of the device, the data processing apparatus is arranged to leave stationary image objects discarded during the calculation of the function in step a). Such stationary image objects may be, for instance, fixed position markers on the patient or patient table whose position is not influenced by the heartbeat or breathing. If such objects were taken into account in, for instance, the aforementioned cross-correlation method, then this would falsify the result. The static image objects to be removed from the calculations may be indicated to the data processing apparatus, for instance interactively, by a user. The data processing apparatus may, however, also be arranged to determine the static image objects automatically by, for instance, comparison of all the angiograms present in the database.

The device also preferably contains a display device, such as a monitor, on which a current image of the body structure and the angiographic image provided by the device may be displayed superimposed. For instance, fluoroscopic images of a catheter in the coronary vessels together with the prepared angiographic image of the coronary vessels may be represented on a monitor.

Furthermore, the device preferably contains an image-forming apparatus for generating the angiograms and/or a current image of the body structure. The image-forming apparatus may, in particular, be an X-ray apparatus and/or an MRI device.

Furthermore, the device preferably contains sensory apparatus with which the heartbeat phase and/or the respiratory phase may be detected. For instance, an electrocardiographic device for determining the electrocardiogram (ECG) is included which displays the electrical heartbeat phase.

The invention also relates to a method for providing an angiographic image of a body structure going with a given heartbeat phase and respiratory phase, based on a database with angiograms of the body structure from different heartbeat phases and respiratory phases. The method comprises the following steps:

a) The calculation of a function which describes (at least) one change in the body structure dependent upon the respiratory phase, whereby the calculation is based on the angiograms in the database.

b) The generation of the angiographic image to be prepared from at least one angiogram of the database, whose heartbeat phase matches the given heartbeat phase, with the aid of the function calculated in step a).

The method, in the general form, implements the steps to be carried out by a device of the type described above. With regard to the details, advantages and further developments of the method, reference is therefore made to the above explanation.

These and other aspects of the invention are apparent from and will be elucidated with reference to the embodiments described hereinafter.

In the drawings:

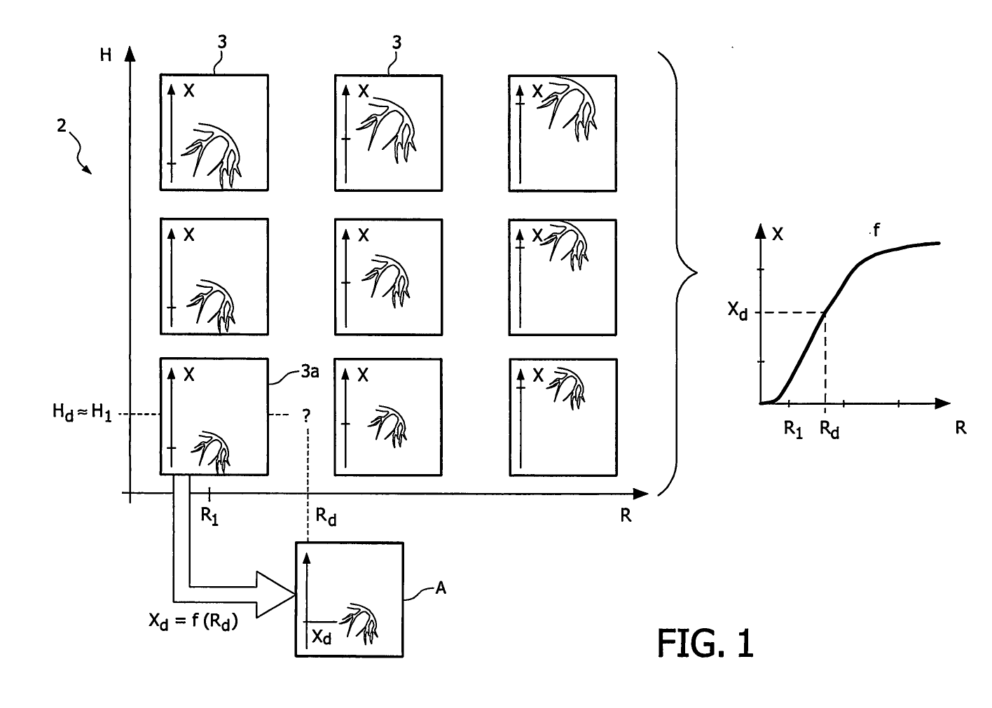

FIG. 1 shows schematically the principle according to the invention for determining an angiographic image;

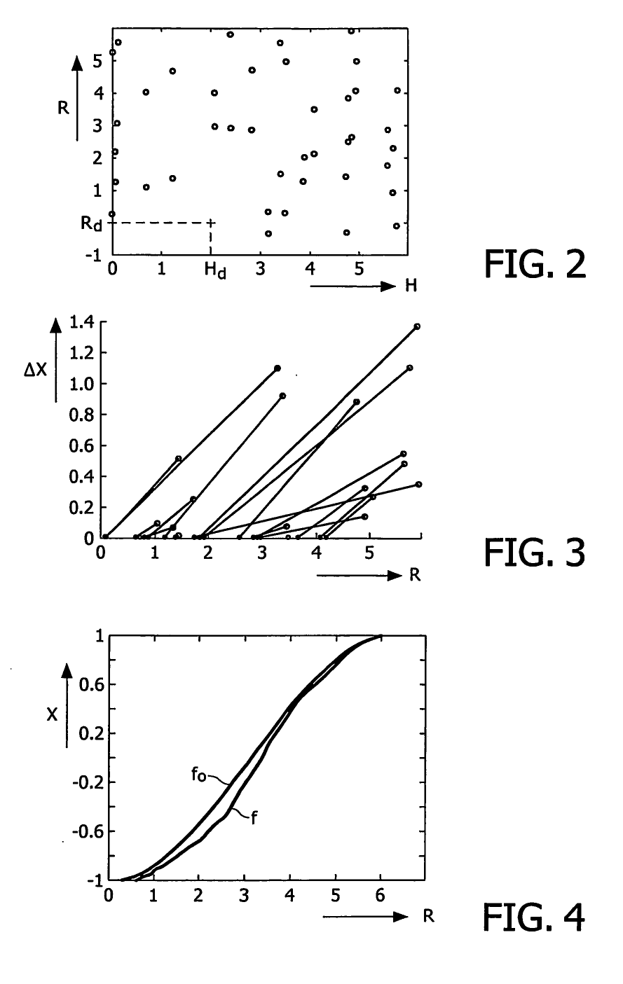

FIG. 2 shows an example of the distribution of angiograms stored in a database over various heartbeat phases and respiratory phases;

FIG. 3 shows the heart displacements calculated from the angiograms of FIG. 2, as a function of the respiratory phase;

FIG. 4 shows a comparison of the unknown function of the heart displacement due to breathing with the function f determined from the angiograms of FIG. 2.

The invention will now be described based on the important application example of a catheter examination of the coronary arteries. In medical interventions of this type, the aim is to navigate a guide wire, balloon or stent on the tip of a catheter as precisely as possible to a site to be treated, such as a stenosis in a coronary vessel. The catheter is moved under constant X-ray fluoroscopic observation. On the associated recordings, however, the vessel system is not visible, since the patient cannot be continuously subjected to contrast medium injections. For this reason, a set of angiograms recorded before or during the intervention are used, which were generated with contrast medium administration and therefore clearly depict the vessel system.

With the methods currently used in catheter laboratories, the current X-ray image is displayed adjacent to a static angiogram, whereby the treating physician has to mnerge mentally the information from the two images. In order to support the physician, it is desirable to represent the static angiogram and the current X-ray image superimposed. Since, however, the heart continuously changes its form and position due to the heartbeat and the respiration, such superpositioning only produces satisfactory results when an angiogram which matches the current image in relation to the heartbeat phase and respiratory phase is used for superpositioning.

In this regard, FIG. 2 shows the distribution of a typical set of 40 angiograms of a database in relation to the respective associated heartbeat phase H and respiratory phase R. As can be seen, the two-dimensional parameter range can only be relatively thinly covered due to the limited number of angiograms. If, therefore, for instance a suitable angiogram is sought for a current fluoroscopic recording from the heartbeat phase H_{d} and the respiratory phase R_{d}, the nearest angiograms of the database are often relatively far form the given data, which leads to a correspondingly erroneous superpositioning.

In order to eliminate this problem, the following method elucidated with the aid of FIG. 1 is proposed. FIG. 1 shows, in the left-hand portion, the database 2 schematically again with the angiograms 3, 3a, . . . it contains, which are represented in a diagram as in FIG. 2 (with swapped axes) corresponding to the associated respiratory phase R and heartbeat phase H. The angiograms 3, 3a show the cardiac vessels 1 as the interesting body structure, whereby in the schematic representation the influence of heart activity is symbolized by a size change in the vessels 1 and the influence of respiration is symbolized by a displacement of the vessels 1 in the x-direction. In practice, it is found that the influence of respiration on the heart may actually approximately be described by a simple displacement of the heart in the direction of the vertical body axis (x).

Firstly, from the angiograrns 3, 3a, . . . available in the database 2, the functional relationship f represented on the right side in FIG. 1, which describes the position x of the heart vessels 1 dependent upon the respiratory phase R, is determined. All the angiograms from all the heartbeat phases and respiratory phases go into the determination of this function f. Details of the determination are explained below by reference to FIGS. 3 and 4.

With the aid of the breathing displacement function f, for a given respiratory phase R_{d}, the associated position x_{d} of the heart may be calculated. Onto one of the angiograms whose heartbeat phase H₁ is the same as the given heartbeat phase H_{d} or comes as close to it as possible, a displacement may be applied which transfers the heart to the position f(R_{d})=x_{d}. In FIG. 1 the angiogram designated 3a may, for instance, be used for this.

By means of an appropriate displacement of the angiogram 3a, therefore, the angiographic image A which is sought and matches the current values of the heartbeat phase H_{d} and the respiratory phase R_{d} in the best way possible is generated. This image A may then, for instance, be displayed superimposed on a current X-ray fluoroscopic image (not shown), whereby a high degree of matching is achieved, permitting the physician comfortable navigation of an intervention instrument.

The angiographic image A may also alternatively be generated in a more complex method by interpolation from a plurality of angiograms with the heartbeat phase H_{d}.

FIG. 3 shows calculated displacements Δx of the heart position between two angiograms, respectively, which belong to the same heartbeat phase, but to different respiratory phases. In each case, two angiograms were selected whose heartbeat phase is the same or very similar, i.e. whose associated points in FIG. 2 lie over one another. The relative displacement Δx between these angiograms was then calculated (see below) and two points were entered in the diagram of FIG. 3 for each angiogram, corresponding to their respiratory phase R, whereby the point for one angiogram lies on the R-axis and the point for the other angiogram lies at the height of the calculated Δx coordinates. Finally, said points were linked by a line in order to indicate their belonging together.

After that, from the data in FIG. 3, the function f(R) represented in FIG. 4 which describes the heart position x dependent upon the respiratory phase may be calculated iteratively. During the iteration, the assumption is made at first that the function f is a constant, that is that it is independent of the respiratory phase R. From this starting point, one data pair of FIG. 3 linked by a line after the other is integrated into the curve. The curve shape is amended for each data pair such that the differences Δx_{f} calculated from the curve f always agree better with the measured differences Δx from FIG. 2. For instance, in the first iteration step, with the integration of a data pair (R1, 0), (R2, Δx) from FIG. 3, the constant function f is amended piece by piece into a new linear function f* such that it gains an increasing linear course between R1 and R2, whereby f*(R2)−f*(R1)=Δx. Further data pairs are, in principle, similarly integrated into the curve, whereby for stabilizing the algorithm, incoming data is asymptotically less weighted than data already integrated into the function. Naturally, other algorithms may also be used for determining the function f being sought, for instance, such as those which minimize the deviation between the differences of the heart position x described by a parametric model function f and the measured differences (FIG. 3) in the heart position.

As a result, what is finally obtained is the curve shape designated as f in FIG. 4, which comes very close to the unknown “true” function f₀. The function f may be used, as explained above in relation to FIG. 1, to transform available angiograms which go with a current heartbeat phase H_{d} but not a current respiratory phase R_{d}, such that the transformed image A goes with both the heartbeat phase and the respiratory phase.

The difference in heart positions on which FIG. 3 is based may be calculated with the aid of methods such as a normalized cross-correlation or maximization of the mutual information (P. Viola, W. M. Wells III: “Alignment by Maximization of Mutual Information”, Int. J. of Computer Vision, 24(2), pp. 137-154 (1997)), since on images from two different respiratory phases but the same heartbeat phase, the heart shows substantially the same form. It should be noted, however, that certain immobile objects (e.g. markers) in the angiograms are excluded from the calculations, since they would falsify the position estimation.

1. A device for providing an angiographic image (A) of a body structure (1) matching a given heartbeat phase (H_{d}) and a respiratory phase (R_{d}), comprising a database (2) with angiograms (3, 3a) of the body structure (1) from different heartbeat phases (H) and respiratory phases (R), and a data processing apparatus linked thereto, which is arranged to carry out the following steps:
a) Calculation of a function (f), which describes a change (x) in the body structure (1) dependent upon the respiratory phase (R), which calculation takes place based on from the angiograms (3, 3a) in the database (2);
b) Generation of the angiographic image (A) to be produced from at least one angiogram (3a) of the database (2), whose heartbeat phase (H₁) matches the given heartbeat phase (H_{d}) with the aid of the calculated function (f).

2. A device as claimed in claim 1, characterized in that the database (2) contains approximately between 10 and 100, and preferably between 30 and 50 angiograms (3).

3. A device as claimed in claim 1, characterized in that the function (f) describes a change in the position of the body structure (1).

4. A device as claimed in claim 1, characterized in that the data processing apparatus is arranged to determine a change in the position of the body structure (1) by a cross-correlation and/or maximization of the mutual information in relation to a reference angiogram.

5. A device as claimed in claim 1, characterized in that the data processing apparatus is arranged to leave static image objects discarded in the calculation of the function (f).

6. A device as claimed in claim 1, characterized in that it includes a display device for superimposed representation of a current image of the body structure (1) and the provided angiographic image (A).

7. A device as claimed in claim 1, characterized in that it includes an image-forming apparatus, in particular an X-ray apparatus and/or an MRI device.

8. A device as claimed in claim 1, characterized in that it includes an electrocardiographic device for determining an electrocardiogram.

9. A device as claimed in claim 1, characterized in that it includes a respiratory phase sensor.

10. A method for providing an angiographic image (A) of a body structure (1) matching a given heartbeat phase (H_{d}) and a respiratory phase (R_{d}), based on a database (2) with angiograms (3, 3a) of the body structure (1) from different heartbeat phases (H) and respiratory phases (R), including the following steps:
a) Calculation of a function (f) which describes a change in the body structure (1) dependent upon the respiratory phase (R), which calculation takes place based on the angiograms (3, 3a) in the database (2);
b) Generation of the angiographic image (A) to be provided from at least one angiogram (3a) of the database (2), whose heartbeat phase (H₁) matches the given heartbeat phase (H_{d}), with the aid of the calculated function (f).
